(19) United States
(12) Patent Application Publication
(10) Pub. No.: US 2024/0082473 A1
(43) Pub. Date: Mar. 14, 2024

(54) MULTI-MICRO-ORGAN EXTRACORPOREAL CIRCULATION AND NEURAL NETWORK MAINTENANCE SYSTEM

(71) Applicants: Yulin CAO, Jiyuan (CN); Tangyi Holding (Shenzhen) Co., Ltd., Shenzhen (CN)

(72) Inventor: Yulin CAO, Beijing (CN)

(73) Assignees: Yulin CAO, Jiyuan (CN); Tangyi Holding (Shenzhen) Co., Ltd., Shenzhen (CN)

(21) Appl. No.: 18/150,813

(22) Filed: Jan. 6, 2023

(30) Foreign Application Priority Data
Sep. 14, 2022 (CN) 202211118478.5

(51) Int. Cl. A61M 1/36

(52) U.S. Cl. CPC A61M 1/3653; A61M 1/3667; A61M 60/113

(57) ABSTRACT

A multi-micro-organ extracorporeal circulation and neural network maintenance system, includes a plurality of organ specificity maintenance systems, and each independent organ specificity maintenance system is connected to a human brain organ specificity maintenance system and a heart organ specificity maintenance system in a two-way manner by means of a simulated neural network. The disclosure innovatively constructs a microphysiological system to simulate in-vitro interaction of complex organs of human blood circulation and nerve regulation and control: through integration of biology and engineering, a traditional organ-like culture mode is broken through, and an extracorporeal circulation system and internal vascular systems of the complex organs are organically integrated and connected to realize perfusion and metabolic circulation; biosensing and signal positive feedback transmission are used for information communication between multi-micro-organs; and through a controllable circulation device, a controllable “switch” is mounted to realize interaction and function regulation and control between any organs.

### CROSS REFERENCE TO THE RELATED APPLICATIONS

This application is based upon and claims priority to Chinese Patent Application No. 202211118478.5, filed on Sep. 14, 2022, the entire contents of which are incorporated herein by reference.

### TECHNICAL FIELD

The disclosure relates to the technical field of extracorporeal circulation and neural network maintenance, and particularly to a multi-micro-organ extracorporeal circulation and neural network maintenance system.

### BACKGROUND

As one form of generalized tissue culture, organ culture means isolated culture of part of a living body. Specifically, part or whole organ is cultured under the condition of not damaging normal tissue structures, that is, a three-dimensional structure of tissue is still maintained, and organ functions in various states are simulated. However, the traditional organ-like culture mode cannot organically integrate and connect the extracorporeal circulation system and the internal vascular systems of the complex organs.

Therefore, we propose a multi-micro-organ extracorporeal circulation and neural network maintenance system to solve the above problems.

### SUMMARY

The objective of the disclosure is to provide a multi-micro-organ extracorporeal circulation and neural network maintenance system, so as to solve the above problems in the background art.

In order to realize the above objective, the disclosure provides the technical solution as follows:
a multi-micro-organ extracorporeal circulation and neural network maintenance system includes a plurality of organ specificity maintenance systems, each independent organ specificity maintenance system is connected to a human brain organ specificity maintenance system and a heart organ specificity maintenance system in a two-way manner by means of a simulated neural network, each independent organ specificity maintenance system is further connected to a one-way venous line by means of simulated venous flow, each independent organ specificity maintenance system is further connected to a one-way arterial line by means of simulated arterial flow, and each independent organ specificity maintenance system is connected to two signal communication nodes.

In a further example, the human brain organ specificity maintenance system is connected to the one-way venous line, such that venous flow in the human brain organ specificity maintenance system enters the one-way venous line; the one-way arterial line is connected to the human brain organ specificity maintenance system, such that arterial flow flowing out of the organ specificity maintenance system flows back into the human brain organ specificity maintenance system; the heart organ specificity maintenance system is connected to the one-way arterial line, such that arterial flow in the heart organ specificity maintenance system enters the one-way arterial line; and the heart organ specificity maintenance system is in communication with the one-way venous line, such that venous flow passing through the heart organ specificity maintenance system flows back into the heart organ specificity maintenance system.

In a further example, the heart organ specificity maintenance system includes a vascularized heart micro-organ.

In a further example, on the one hand, the one-way arterial line is connected to a metabolic product by means of simulated arterial flow and a micro-flow pump, and on the other hand, the one-way arterial line is connected to a micro-organ specificity bioreactor by means of simulated arterial flow, a micro-flow pump and a micro-organ circulation coupling device.

In a further example, the human brain organ specificity maintenance system includes a vascularized human brain micro-organ.

In a further example, the one-way venous line and the one-way arterial line are both connected to a plurality of sensor integrated microfluidic chips.

In a further example, the plurality of organ specificity maintenance systems may include a liver and gall portion, an ovary portion, a skin portion, an expanded organ portion, etc.

Compared with the prior art, the disclosure has the beneficial effects:
the disclosure innovatively constructs a microphysiological system to simulate in-vitro interaction of complex organs of human blood circulation and nerve regulation and control: through integration of biology and engineering, a traditional organ-like culture mode is broken through, and an extracorporeal circulation system and internal vascular systems of the complex organs are organically integrated and connected to realize perfusion and metabolic circulation; biosensing and signal positive feedback transmission are used for information communication between multi-micro-organs; and through a controllable circulation device, a controllable “switch” is mounted to realize interaction and function regulation and control between any organs.

### BRIEF DESCRIPTION OF THE DRAWINGS

FIG. 1 is a relation construction diagram of a multi-micro-organ extracorporeal circulation and neural network maintenance system in the disclosure; and

FIG. 2 is an enlarged view of position A in FIG. 1.

### DETAILED DESCRIPTION OF THE EMBODIMENTS

The technical solutions of the examples of the disclosure are clearly and completely described below in combination with the accompanying drawings in the examples of the disclosure. Apparently, the described examples are only some examples rather than all examples of the present disclosure. On the basis of the examples of the disclosure, all other examples obtained by a person of ordinary skill in the art without making creative efforts all fall within the scope of protection of the disclosure.

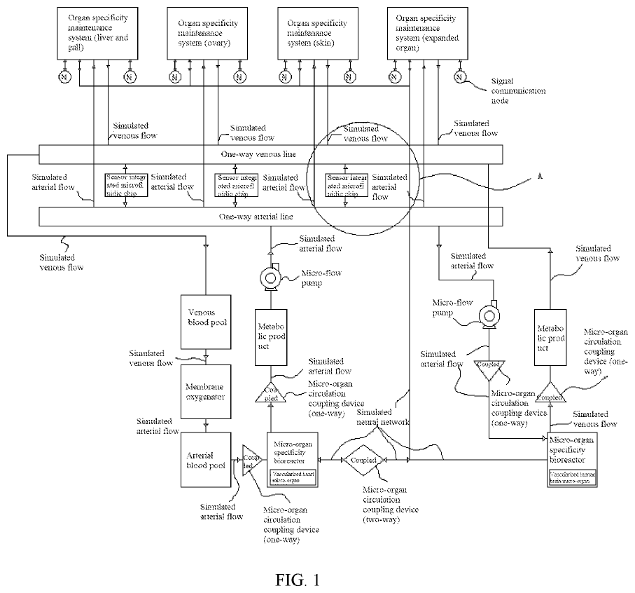

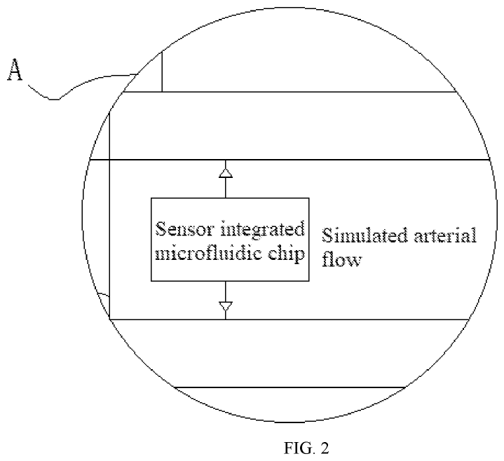

With reference to FIGS. 1 and 2, a multi-micro-organ extracorporeal circulation and neural network maintenance system includes a plurality of organ specificity maintenance systems, each independent organ specificity maintenance system is connected to a human brain organ specificity maintenance system and a heart organ specificity maintenance system in a two-way manner by means of a simulated neural network, each independent organ specificity maintenance system is further connected to a one-way venous line by means of simulated venous flow, each independent organ specificity maintenance system is further connected to a one-way arterial line by means of simulated arterial flow, and each independent organ specificity maintenance system is connected to two signal communication nodes. The human brain organ specificity maintenance system is connected to the one-way venous line, such that venous flow in the human brain organ specificity maintenance system enters the one-way venous line; the one-way arterial line is connected to the human brain organ specificity maintenance system, such that arterial flow flowing out of the organ specificity maintenance system flows back into the human brain organ specificity maintenance system; the heart organ specificity maintenance system is connected to the one-way arterial line, such that arterial flow in the heart organ specificity maintenance system enters the one-way arterial line; and the heart organ specificity maintenance system is in communication with the one-way venous line, such that venous flow passing through the heart organ specificity maintenance system flows back into the heart organ specificity maintenance system. The heart organ specificity maintenance system includes a vascularized heart micro-organ.

On the one hand, the one-way arterial line is connected to a metabolic product by means of simulated arterial flow and a micro-flow pump, and on the other hand, the one-way arterial line is connected to a micro-organ specificity bioreactor by means of simulated arterial flow, a micro-flow pump and a micro-organ circulation coupling device. The human brain organ specificity maintenance system includes a vascularized human brain micro-organ. The one-way venous line and the one-way arterial line are both connected to a plurality of sensor integrated microfluidic chips. The plurality of organ specificity maintenance systems may include a liver and gall portion, an ovary portion, a skin portion, an expanded organ portion, etc.

For those skilled in the art, it is apparent that the disclosure is not limited to the details of the above-mentioned exemplary examples, and the disclosure can be implemented in other specific forms without departing from the spirit or basic features of the disclosure. Therefore, no matter from which point of view, the examples should all be regarded as exemplary and non-limiting. The scope of the disclosure is defined by the appended claims rather than the above-mentioned description, and therefore it is intended that all changes which fall within the meaning and scope of equivalency of the claims are embraced in the disclosure. Any reference numeral in the claims should not be construed as limiting the related claims.

In addition, it should be understood that although the description is described in terms of embodiments, not every embodiment only includes an independent technical solution. The description manner of the description is only for clarity, and those skilled in the art should take the description as an entirety. The technical solutions in various examples can also be appropriately combined to form other embodiments that can be understood by those skilled in the art.

What is claimed is:

1. A multi-micro-organ extracorporeal circulation and neural network maintenance system, comprising:
a plurality of organ specificity maintenance systems, wherein each independent of the plurality of organ specificity maintenance systems is connected to a human brain organ specificity maintenance system and a heart organ specificity maintenance system in a two-way manner by a simulated neural network, each independent of the plurality of organ specificity maintenance systems is further connected to a one-way venous line by a simulated venous flow, each independent of the plurality of organ specificity maintenance systems is further connected to a one-way arterial line by a simulated arterial flow, and each independent of the plurality of organ specificity maintenance systems is connected to two signal communication nodes.

2. The multi-micro-organ extracorporeal circulation and neural network maintenance system according to claim 1, wherein the human brain organ specificity maintenance system is connected to the one-way venous line, such that a venous flow in the human brain organ specificity maintenance system enters the one-way venous line; the one-way arterial line is connected to the human brain organ specificity maintenance system, such that an arterial flow flowing out of the plurality of organ specificity maintenance systems flows back into the human brain organ specificity maintenance system; the heart organ specificity maintenance system is connected to the one-way arterial line, such that an arterial flow in the heart organ specificity maintenance system enters the one-way arterial line; and the heart organ specificity maintenance system is in a communication with the one-way venous line, such that a venous flow passing through the heart organ specificity maintenance system flows back into the heart organ specificity maintenance system.

3. The multi-micro-organ extracorporeal circulation and neural network maintenance system according to claim 1, wherein the heart organ specificity maintenance system comprises a vascularized heart micro-organ.

4. The multi-micro-organ extracorporeal circulation and neural network maintenance system according to claim 3, wherein the one-way arterial line is connected to a metabolic product by the simulated arterial flow and a micro-flow pump, and the one-way arterial line is connected to a micro-organ specificity bioreactor by the simulated arterial flow, the micro-flow pump and a micro-organ circulation coupling device.

5. The multi-micro-organ extracorporeal circulation and neural network maintenance system according to claim 4, wherein the human brain organ specificity maintenance system comprises a vascularized human brain micro-organ.

6. The multi-micro-organ extracorporeal circulation and neural network maintenance system according to claim 1, wherein the one-way venous line and the one-way arterial line are both connected to a plurality of sensor integrated microfluidic chips.

7. The multi-micro-organ extracorporeal circulation and neural network maintenance system according to claim 1, wherein the plurality of organ specificity maintenance systems are allowed to comprise a liver and gall portion, an ovary portion, a skin portion, and an expanded organ portion.
